(19) United States
(12) Patent Application Publication
(10) Pub. No.: US 2009/0236259 A1
(43) Pub. Date: Sep. 24, 2009

(54) Sterile vascular access kit

(76) Inventor: Lois A. Hicks, Elizabethtown, PA (US)

Correspondence Address: LITMAN LAW OFFICES, LTD., POST OFFICE BOX 15035, CRYSTAL CITY STATION, ARLINGTON, VA, 22215-0035, US

(21) Appl. No.: 12/382,484

(22) Filed: Mar. 17, 2009

Related U.S. Application Data
(60) Provisional application No. 61/064,683, filed on Mar. 19, 2008.

(51) Int. Cl. A61B 19/02

(52) U.S. Cl. 206/571; 206/570

(57) ABSTRACT

The sterile vascular access kit contains sterile supplies needed to access an implanted vascular device, such as a Mediport. The kit contains all the medical supplies needed to accomplish the vascular access procedure, thereby eliminating the need to locate and gather such medical supplies. The medical supplies include a facemask and gloves, Tegaderm dressing for providing a sterile covering, a needleless blood transfer device, an injection cap, syringes containing normal saline for flushing the tubing, and empty syringe for drawing blood, preferably a syringe of heparin for flushing the tubing and port. The kit also contains sponges, sterile adhesive strips, alcohol prep pads and Chloraprep for cleansing the access site.

### CROSS-REFERENCE TO RELATED APPLICATION

This application claims the benefit of U.S. Provisional Patent Application Ser. No. 61/064,683, filed Mar. 19, 2008.

### BACKGROUND OF THE INVENTION

1. Field of the Invention

The present invention generally relates to medical devices, and more particularly to a sterile vascular access kit containing sterile supplies for use in accessing a Mediport or implanted central venous catheter in order to draw blood or to administer medications, such as antibiotics, chemotherapy, IV fluids and blood products.

2. Description of the Related Art

Many patients have conditions that require the administration of IV fluids or require frequent blood testing of various labs, such as complete blood count, electrolyte levels, medication levels, etc., to determine that therapeutic medication dosages have been achieved and the patient ready for continued treatments or changes in their care plan. Instead of accessing a new IV site every time fluid administration or blood testing is required, such patients have a surgically implanted central venous device such as a Mediport placed.

While such devices simplify access to the vein, the port presents a potential site where infection may develop whenever the port is accessed. In order to reduce the risk of infection, and to ensure the proper administration of fluids or taking of blood samples, the nurse or health care provider requires several medical appliances or items of equipment. The process of gathering the equipment may take time, and when the health care provider is pressed for time, there may be a temptation to use appliances that have not been properly sterilized, or to omit some steps entirely if the equipment is not readily at hand. There is also the potential to contaminate a sterile field during set up of supplies by opening many individual contents and placing them on the field as well as drawing up Saline and Heparin with sterile technique. This situation may be frequently encountered in hospitals, in nursing homes, among emergency medical technicians, and among visiting home health care practitioners.

Thus, a sterile vascular access kit solving the aforementioned problems is desired.

### SUMMARY OF THE INVENTION

The sterile vascular access kit contains sterile supplies needed to access an implanted vascular device, such as a Mediport, which is employed to obtain blood samples and for giving frequent intravenous (IV) medication over a period of time, up to seven days before changing the access needle. As indicated above, the kit contains all the medical supplies needed to accomplish the vascular access procedure, thereby eliminating the need to locate and gather such medical supplies.

The medical supplies include a facemask and gloves, compatible for administering chemotherapy. Tegaderm/sterile occlusive dressing for providing an enclosure over the needle accessed site, a needleless fluid transfer device, an injection cap, syringes containing normal saline for flushing the tubing, an empty syringe for drawing blood, and preferably a syringe of Heparin or anticoagulant for flushing the tubing and implanted central line after each use and prior to each de-access of the implanted device. The kit also contains sponges, sterile adhesive strips, and Chloraprep for cleansing the site of access.

Accordingly, the invention presents an all-inclusive vascular access kit that is compactly designed and sealed to prevent contamination of the supplies contained therein. The invention provides for improved elements thereof in an arrangement for the purposes described that are inexpensive, dependable and fully effective in accomplishing their intended purposes.

These and other features of the present invention will become readily apparent upon further review of the following specification and drawings.

### BRIEF DESCRIPTION OF THE DRAWINGS

FIG. 1 is a perspective view of a sterile vascular access kit according to the present invention.

FIG. 2 is a detailed, perspective view of the contents of a sterile vascular access kit according to the present invention.

Similar reference characters denote corresponding features consistently throughout the attached drawings.

### DETAILED DESCRIPTION OF THE PREFERRED EMBODIMENTS

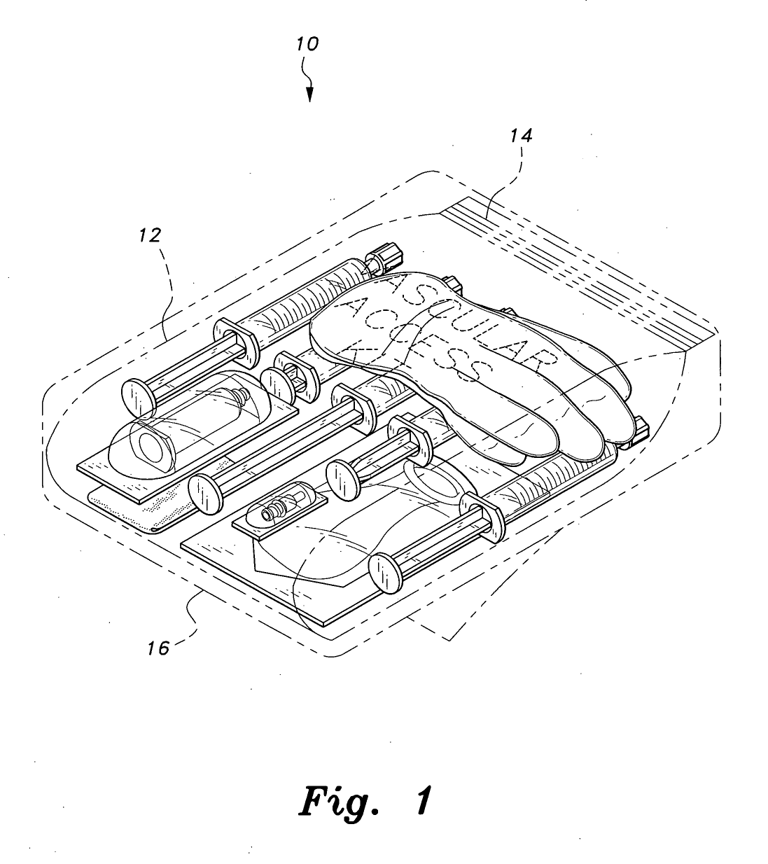

Attention is first directed to FIG. 1, wherein a sterile vascular access kit is generally indicated at 10. The kit 10 includes a storage tray 12 housing all essential medical items needed to access an implanted venous device. Tray 12 is wrapped in a blue CSR (Central Service Room) wrap 16 and further enclosed in an easily removable, fluid-impervious sealing material 14. Kit 10 is compact and can be conveniently stored.

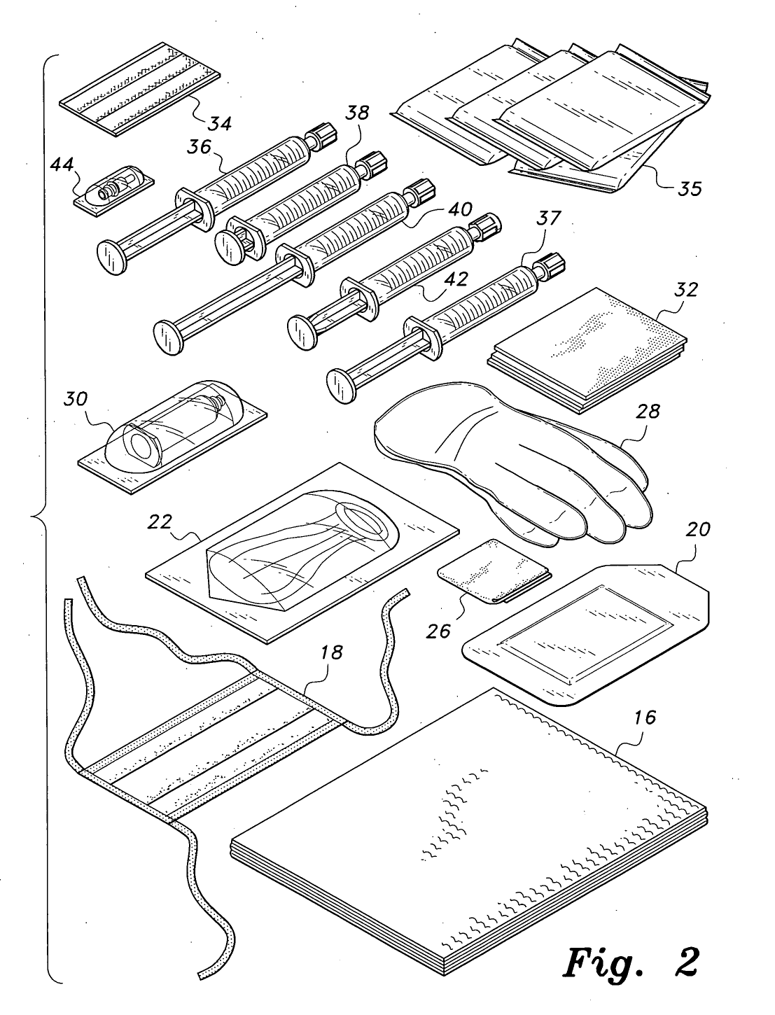

The contents of kit 10 are best shown in FIG. 2. The contents include the above-mentioned blue CSR wrap 16, a facemask 18, a 4″×4¾″ Tegaderm™ dressing 20 (Tegaderm is a product of Minnesota Mining & Manufacturing [3M] Company of St. Paul, Minn., referring to a polyurethane film sheet with an adhesive layer, the sheet being porous to air but providing a barrier against bacterial infection), ChloraPrep Onestep 22 and two (2″×2″), non-woven, split, sponges 26. A pair of wrapped, cuffed latex or other suitable gloves 28 is also available in the kit. Other essential items are a needleless blood or fluid transfer device 30, two (4″×4″) non-woven cover sponges 32, three (½″×4″) steri strips 34, a needleless injection cap 44, and four alcohol prep pads 35.

There are five syringes included in the kit 10. Two 10 ml syringes 36 and 40, each contain 10 ml of normal saline solution, which is the recommended flushing protocol for adult Mediports. One syringe 37 contains only 5 ml normal saline solution used to prime the tubing of the access needle. An empty syringe 38, e.g., a 10 ml syringe, is available for collecting a blood sample. The last syringe 42 is made available to contain an anticoagulant, such as heparin, for flushing the port. Preferably, a 10 ml syringe containing about 3 ml of heparin (1:100 units/ml-pediatric dose) or 5 ml (1:100 units/ml-adult dose) is included in the kit 10 if the heparin can be kept stable during the expected storage period. Otherwise, a second empty 10 ml syringe may be provided for the administration of heparin. The heparin would need to be drawn up with one-handed sterile technique or have a second health care worker assist to assure the heparin is prepared for use in a sterile manner on the tray prior to access.

Variations in contents can be made, if necessary. A kit 10 could function for the purpose of lab draws and port flushing only by not providing the steri strips and Tegaderm/occlusive dressing. Other variations would include additional components for servicing a double lumen port. Such additional components might include an additional three steri strips, additional 2″×2″ split sponges, an additional injection cap, additional syringes (one each of those enumerated above), and an additional or larger size Tegaderm dressing.

In use, the health care practitioner maintains a sterile field by opening the blue CSR wrap and then places an appropriate packaged sterile sized needle for access onto the CSR (sterile field). The health care practitioner will then put on the mask and sterile gloves. The 10 ml syringe with 5 mls of saline will be used to flush the needle and attached tubing. The 10 ml syringe with 10 mls of saline will be attached to the injection cap and flushed with a small quantity of saline. All the supplies in the tray will be assembled for easy reach on the CSR sterile field for the health care practitioner.

The area over the implanted central line/Mediport is cleansed with Chloraprep Onestep for 30 seconds and allowed to air dry. A 4×4 is opened and placed just below the area cleansed to act as a sterile site to rest tubing if needed. The needle is then used to access the port. The partially filled syringe of saline is still attached and drawn back to the 10 ml mark. This is the discard sample of blood, as it is mixed with the previous flush that has been sitting in the line between uses. Place the syringe in the tray for discard.

The empty syringe is attached next, making sure to expel all air from the syringe before attaching and clamping the tubing between each change of syringes. Draw the required blood sample and place the syringe on a corner of the sterile field. Attach the 10 ml saline syringe connected to the injection cap and flush tubing. There should be little to no resistance met. Cleanse the injection cap and administer a second 10 ml saline flush as required per protocol when indicated for the Mediport and administer appropriate dose of heparin flush to the patient. The 2×2 split sponge may be placed under/around the needle device as a cushion and steri strips applied over the sponges and needle in a crisscross fashion to secure the needle.

The Tegaderm/occlusive dressing is then applied over the needle, 2×2 and steri strips. This acts as a barrier against potential organisms entering the site of access to the patient. The syringe holding the blood sample should now be attached to the blood transfer device and placed in the required lab collection tubes. The waste supplies may then be disposed of in a sanitary manner. It will be noted that the tubing is clamped between each change of syringe.

It will be seen that the kit elements cooperate to provide for sterile vascular access to the Mediport. It is to be understood that the present invention is not limited to the embodiments described above, but encompasses any and all embodiments within the scope of the following claims.

I claim:

1. A sterile vascular access kit, comprising:
a storage tray;
means housed in the storage tray for performing a sterile vascular access procedure; and
a blue CSR wrap enclosing the storage tray and the means for performing the sterile vascular access procedure.

2. The sterile vascular access kit according to claim 1, further comprising sealing material further enclosing said sterile vascular access kit.

3. A sterile vascular access kit, comprising:
a storage tray;
means housed in the storage tray for performing a sterile vascular access procedure
a blue CSR wrap enclosing the storage tray and the means for performing the sterile vascular access procedure; and
a fluid-impervious sealing material further enclosing the sterile vascular access kit.

4. The sterile vascular access kit according to claim 3, wherein said means for performing a sterile vascular access procedure comprises:
a facemask and sterile gloves adapted to be donned by a health care practitioner;
a sealed Tegaderm™ dressing;
a sealed ChloraPrep Onestep;
a plurality of non-woven, split sponges;
a needleless fluid transfer device;
a plurality of non-woven cover sponges;
a plurality of steri strips;
a needleless injection cap;
a plurality of alcohol prep pads; and
a plurality of syringes.

5. The sterile vascular access kit according to claim 4, wherein at least two of said plurality of syringes, respectively contain 10 ml of normal saline solution and one of said syringes contains 5 ml of normal saline solution.

6. The sterile vascular access kit according to claim 4, wherein at least one of said plurality of syringes is empty.

7. The sterile vascular access kit according to claim 4, wherein at least one of said plurality of syringes contains an anticoagulant.

8. A sterile vascular access kit, comprising:
a storage tray;
means housed in the storage tray for performing a sterile vascular access procedure, the means including;
a facemask and sterile gloves adapted to be donned by a health care practitioner;
a sealed Tegaderm™ dressing;
a sealed ChloraPrep Onestep;
a plurality of non-woven, split sponges;
a needleless fluid transfer device;
a plurality of non-woven cover sponges;
a plurality of steri strips;
a needleless injection cap;
a plurality of alcohol prep pads; and
at least five syringes;
a blue CSR wrap enclosing the storage tray and the means for performing the sterile vascular access procedure; and
a fluid-impervious sealing material further enclosing the sterile vascular access kit.

9. The sterile vascular access kit according to claim 8, wherein at least two of said at least five syringes, respectively contain 10 ml of normal saline solution and one of said at least five syringes contain 5 ml of normal saline solution.

10. The sterile vascular access kit according to claim 9, wherein at least one of said at least five syringes is empty.

11. The sterile vascular access kit according to claim 10, wherein at least one of said at least five syringes contains an anticoagulant.
